(19) United States
(12) Patent Application Publication
(10) Pub. No.: US 2017/0209547 A1
(43) Pub. Date: Jul. 27, 2017

(54) NUTRITIONAL SUPPLEMENT FOR USE IN COMBATTING STRESS AND ANXIETY IN PETS

(71) Applicant: DOMES PHARMA, Paris (FR)

(72) Inventor: Anne CHAUDER, Clermont-Ferrand (FR)

(73) Assignee: DOMES PHARMA, Paris (FR)

(21) Appl. No.: 15/411,539

(22) Filed: Jan. 20, 2017

(30) Foreign Application Priority Data
Jan. 21, 2016 (FR) 1650472

(51) Int. Cl. A61K 38/38; A61K 31/661; A61K 9/00; A61K 33/06

(52) U.S. Cl. CPC A61K 38/38; A61K 33/06; A61K 31/661; A61K 9/0053

(57) ABSTRACT

Nutritional supplement for veterinary, for use in combatting stress and anxiety in pets, characterized in that it contains a mixture of α-lactalbumin and magnesium.

The present invention relates to a veterinary composition to combat stress and anxiety in pets. More specifically, it relates to a nutritional supplement intended for cats, dogs and exotic pets with a view to calming anxiety and moderate states of stress in these animals.

In pets, particularly cats, dogs and exotic pets, encountering states of stress or chronic or temporary anxiety is quite frequent.

This stress or anxiety may have different origins and trigger factors. They are for example often related to the animal's solitude and captivity conditions; it may lack activity or stimulation (animals left alone, kept in apartments, shut in or attached), to changes in habits or living conditions (a person or animal arriving or leaving, moving house, work in the house, or changes of furniture or decoration, etc.), to separation from its owner, to phobias or fears relating to an unusual situation (ear-piercing noises, storms, fireworks, car transport, veterinary visits, etc.).

A state of stress or anxiety like this may manifest in different ways depending on the animal, and in particular through behavioral problems and/or clinical manifestations. As examples, excessive barking or meowing; hyperactivity and aggression; urinary or fecal uncleanliness; excessive and compulsive licking; panting, perspiration and trembling; diarrhea and vomiting; destruction of the surrounding environment and self-mutilation may be cited.

Such behavior is often difficult for owners to bear and can cause rapid deterioration in relations between the pet and its owner. In addition, in the long term, stress and anxiety may give rise to health problems in the animal that may have serious consequences, such as gastrointestinal, dermatological or urinary problems, a predisposition to infections, anorexia or bulimia, etc.

The purpose of the invention is to propose a nutritional supplement that effectively combats moderate stress and anxiety in pets and in particular in cats, dogs and exotic pets, such as for example rabbits, hamsters, guinea pigs, rats, mice and other rodents, ferrets, etc.

To solve the technical problem, the invention teaches a nutritional supplement for veterinary use, intended to combat stress and anxiety in pets, which is characterized in that it contains a mixture of α-lactalbumin and magnesium.

Preferably, the nutritional supplement according to the invention contains magnesium in the form of magnesium glycerophosphate and/or magnesium stearate.

According to the invention, the nutritional supplement contains mainly, as a magnesium source, magnesium glycerophosphate. Indeed, the inventors have noticed that magnesium glycerophosphate had high bioavailability and a less bitter taste than other forms of magnesium. In practice, magnesium glycerophosphate represents at least 80%, advantageously more than 90% by weight of the magnesium source.

According to a preferred embodiment of the invention, the nutritional supplement contains more than 50%, and preferably about 70% by weight of a mixture of α-lactalbumin and magnesium. In this mixture, the α-lactalbumin/magnesium ratio is advantageously comprised between 50/50 and 80/20, preferably of the order of 70/30.

According to a preferred embodiment of the invention, the nutritional supplement further contains at least one palatable excipient.

The nature of this palatable excipient is preferentially chosen as a function of the animal species for which the nutritional supplement is intended. It may vary according to the applications envisaged. It is in particular a derivative of animal by-products.

According to one embodiment of the invention, the nutritional supplement further comprises one or more compounds or excipients chosen from binders, absorbents, suspension agents, diluents, compression excipients, cellulose derivatives, microcrystalline cellulose and calcium phosphate.

According to one embodiment of the invention, the dose used is between 15 mg and 60 mg of nutritional supplement per kilo of weight of the animal concerned. It is preferentially 25 mg or 50 mg of nutritional supplement per kilo of weight of the animal concerned.

The nutritional supplement is intended to be administered orally. For this, it can be presented in different pharmaceutical forms, for example in tablet form, as a soft chew, as a powder packaged as capsules, as a liquid to pour on food or in drinking water, or any other appropriate form.

According to a preferred embodiment of the invention, the nutritional supplement is presented in the form of a powder packaged in a capsule. The powder is intended to be dispersed onto the animal's dry or wet food to then be ingested.

The dosage advised is for example one capsule per day for 5 kg of animal weight. The nutritional supplement is preferentially provided to be administered as a one month treatment, which may be extended. It can advantageously be sold in a box of 30 capsules, which corresponds to the quantity necessary for a one month treatment for an average cat or a small dog.

The nutritional supplement according to the invention is effective and has a calming action without generating notable side effects, unlike classic antidepressant drugs that are often responsible for addiction and drowsiness.

In effect, in the event of stress, the serotonin present in the subject's cortex is consumed in order to produce proteins that makes it possible for a return to a state of calm. Animals that present a state of chronic anxiety or stress have a low serotonin concentration in the cortex due to its continuous consumption, which causes a constant lack of serotonin in the organism and prevents a return to normality.

Since serotonin does not pass through the intestinal barrier, it is not possible to replace this lack by directly introducing serotonin to the food of stressed animals.

Within the body, serotonin is made in several steps by specific neurons in the brain stem from an amino acid called tryptophan. Providing tryptophan through feeding may increase the serotonin concentration in the brain.

However, for tryptophan to reach the brain in sufficient quantity, many difficulties must be overcome: the food provided must be sufficient; the intestinal assimilation must be satisfactory; the tryptophan must not be diverted to other pathways and, finally, it must be absorbed within the brain.

All of this requires a subtle balance of intake of different amino acids.

The α-lactalbumin contained in the nutritional supplement according to the invention is a dietary protein that is very high in tryptophan (it contains about 6% of it). Its contribution, by means of the food supplement of the invention, to the feed of the animals concerned, makes it possible to increase the tryptophan/large neutral amino acid ratio. In this way, tryptophan has an advantage over the competition for reaching the brain and serotonin secretion is favored.

In addition, it has been demonstrated that a lack of magnesium leads to an increase in anxious and depressive behavior in animals. The magnesium contained in the nutritional supplement according to the invention, which is an inorganic inhibitor for NMDA receptors (N-methyl-D-aspartate), has anxiolytic activity comparable to an antidepressant that potentiates the α-lactalbumin according by means of a synergistic effect.

The invention and its advantages will be seen clearly in the following example.

### 1/ Animals and Experimental Groups

Forty male Wistar Han rats (Crl:WI(Han), Charles River Laboratories, L'Arbresle, France), weighing from 250 to 275 g were used. Upon receipt, the rats were marked and split into groups of 3 or 4 animals in polycarbonate cages (Eurostandard type IIIH, 425×266×185 mm, Tecniplast, Lyon, France). The animals were housed in a climate-controlled animal house, at a temperature of 22±202° C. and relative humidity of 50±20%. The rats had food (Teklad Global 2016, Harlan, Ganat, France) and drink ad libitum and were held in a day-night cycle inverted by 12 hours (day from 20:00 to 08:00).

After a one-week laboratory condition familiarization period, the rats were 25 weighed and split randomly into 4 treatment groups (n=10/group):
Group 1 (Vehicle): spring water administered p.o. 60 minutes (min) before the test;
Group 2 (DZ): Diazepam administered at the dose of 3 mg/kg p.o. 60 min before the test;
Group 3 (I-25): Product of the Invention administered at the dose of 25 mg/kg p.o. 60 min before the test;
Group 4 (I-50): Product of the Invention administered at the dose of 50 mg/kg p.o. 60 min before the test.

To prevent any interference between the various treatments, the rats in a given cage all received the same treatment at the same dose. The rats in the different groups were all handled in the same manner and under the same conditions.

### 2/ Test Products and Vehicle

Products 1-25 and I-50 correspond to the nutritional supplement of the invention containing α-lactalbumin (130 mg) and magnesium glycerophosphate (50 mg). The products were dissolved in spring water (Cristaline, Metzeral, France) at concentrations of 2.5, 5 and 10 mg/mL. The spring water was also used as a treatment for the Vehicle group.

Diazepam as a drinkable solution (Valium 1%, Roche, Boulogne-Billancourt, France; Lot No.: M0970M1-12/2013) was suspended in an aqueous solution containing 0.5% methylcellulose (Tylose® MH 300, Sigma-Aldrich, St Quentin Fallavier, France) at a concentration of 0.3 mg/mL.

The treatments were prepared immediately prior to use and administered at a volume of 10 mL/kg.

3/ Study on the Anxiolytic Effects of Product I Administered Orally at the Doses of 25 and 50 mg/kg, in the EDC Test in Adult Wale Wistar Rats

The test took place in 2 phases spread over 3 days (D):
a phase of habituation to test conditions (D1 and D2)
a test phase (D3).

The rats received their treatment on D3, the day of the anxiety test. All of the procedures were carried out blind.

### 3.1—Treatment Administration

On D3, each rat received its treatment orally using a syringe connected to an intragastric feeding tube (Popper & Sons, Inc., New York, USA) 60 min before being tested in the EDC device.

| | | | | | Time of |
|---|---|---|---|---|---|
| | No. | | Dose | | administration |
| | of | | (mg/kg, | Volume | (mm before |
| Group | rats | Product tested | p.o.) | (mL/kg) | the test) |
| Vehicle | 10 | Spring water | — | 10 | 60 |
| Diazepam | 10 | Diazepam + 0.5% | 3 | 10 | 60 |
| | | methycellulose | | | |
| I-25 | 10 | I-25 + spring water | 25 | 10 | 60 |
| I-50 | 10 | I-50 + spring water | 50 | 10 | 60 |

### 3.2—EDC Test

### Experimental Device

The EDC device is composed of a transparent polycarbonate box (44×28×18 cm), whose base has been covered with a layer of sawdust 5 cm thick. A hole that allows an electric probe to be attached two centimeters above the sawdust is present in the center of one of the lateral faces. The electric probe is made of a block of Plexiglas (7×2×0.5 cm) covered with copper wire. The probe is connected to an electric shock generator with a manual trigger (OPEN-Systems, Maxéville, France).

The experimental device was placed in a room lit with a low intensity red light. A video camera connected to a digital recorder saved all behavioral sequences so that behavior of interest could be measured later. From a control monitor placed in an adjacent room an operator administered the electric shocks to each animal and controlled the testing.

### Habituation to Test Conditions

The 2 days before the test (D1 and D2), the rats in a given cage were transported to the animal house in the experimentation room and placed together in the experimental device without the electrode for 20 minutes. Between the habituation period for each cage of rats, the sawdust was changed and evened out to a uniform height of 5 cm. The animals were then brought into the animal house.

### Anxiety Test

The anxiety test took place during the first hours of the night phase of the light cycle corresponding to the animals' period of maximum activity.

The electric probe was inserted into the cage before the start of the test. Each rat was placed individually in the experimental device on the side opposite the probe. A single low-intensity electric shock (2 mA) was delivered to the animal when it put its rear paws on the probe for the first time. After the electric shock, the rat's behavior was recorded for 5 min. The sawdust was evened out to a uniform height of 5 cm before each rat passed through and changed before the test session for each new cage of rats to prevent any interference between the groups.

### Behavioral Variables Studied

The following behaviors were quantified from video recordings made during the tests:
duration probe was buried;
number of stretches in the direction of the probe;
number of approaches to the probe;
number of flights from the probe.

From the approach and flight variables the percentage of approaches followed by flights was calculated: (number of flights/number of approaches)×100. The “burying duration”, “number of stretches” and “percentage of approaches followed by flights” were used to establish the global anxiety score.

For this, a rank was attributed to each rat for each of the 3 variables taken into account. To attribute ranks for a given variable, the rats were classified in increasing order on the basis of this variable. The animal with the lowest value received rank “1”, and the next animal rank “2”, and so on until the animal with the highest value, who received the rank “N”, equal to the total number of rats in the study. If several rats have the same value, each of these rats received the average rank of the series of rats concerned (for example, if the 5 rats with the lowest values are equal—they collectively occupy ranks 1 to 5—each of these rats received the average rank of the series: (1+2+3+4+5)/5=3).

The global anxiety score for each rat corresponds to the sum of the ranks that was attributed to it for the 3 variables taken into account.

### 3.3—Animal Monitoring and Euthanasia

The animals were weighed 4 days after they arrived in the laboratory, and on the test day (D3). The well-being and viability of the animals were checked daily during the entire duration of the study. The animals were euthanized at the end of the study by intraperitoneal injection of an overdose (110 mg) of sodium pentobarbital (Ceva Santé Animale, Libourne, France).

### 3.4—Statistical Analysis

The effect of treatments on the global anxiety score for the rats was analyzed using the Kruskal-Wallis test, followed by the Mann-Whitney U test for multiple comparisons. The significance threshold was set at 5%.

Statistical and graphical processing was implemented using Statview® 5 (SAS Institute, Inc., USA) software programs.

### 4—Results

The Kruskal-Wallis test showed significant heterogeneity among global anxiety scores for rats in different treatment groups [H(ddl=4)=13.069; P=0.009].

The Mann-Whitney test showed that the global anxiety scores for rats in the Diazepam, I-25 and I-50 groups were significantly lower than those for rats in the vehicle group.

| | Group | | | |
|---|---|---|---|---|
| | Vehicle | Diazepam | I-25 | I-50 |
| | (n = 10) | (n = 9) | (n = 10) | (n = 10) |
| Median | 111 | 41 | 55 | 46 |
| QI-QS | 69-124 | 41-80 | 41-80 | 41-54 |

| | | | |
|---|---|---|---|
| Mann-Whitney Test | U = 12 | U = 23 | U = 15 |
| (vs. Vehicle) | P = 0.006 | P = 0.041 | P = 0.008 |

1. A nutritional supplement for use in combatting stress and anxiety in pets, comprising a mixture of α-lactalbumin and magnesium.

2. The nutritional supplement according to claim 1, comprising magnesium glycerophosphate and/or magnesium stearate as a source of the magnesium.

3. The nutritional supplement according to claim 2, comprising, as a magnesium source, magnesium glycerophosphate.

4. The nutritional supplement according to claim 1, containing more than 50% by weight of a mixture of α-lactalbumin and a source of the magnesium.

5. The nutritional supplement according to claim 4, wherein the α-lactalbumin/magnesium source ratio is between 50/50 and 80/20.

6. The nutritional supplement according to claim 1, additionally comprising at least one palatable excipient.

7. The nutritional supplement according to claim 1, further comprising one or more compounds or excipients chosen from binders, absorbents, suspension agents, diluents, compression excipients, cellulose derivatives, microcrystalline cellulose and calcium phosphate.

8. The nutritional supplement according to claim 1, in the form of a tablet, a soft chew, a powder packaged in a capsule, or a liquid to pour over food or into drinking water.

9. The nutritional supplement according to claim 1, in a form suitable for oral use.

10. A method of reducing anxiety in an animal, comprising administering to the animal an effective amount of the nutritional supplement according to claim 1.

11. The nutritional supplement according to claim 3, wherein the magnesium glycerophosphate makes up least 80% by weight of total magnesium source present in the supplement.

12. The nutritional supplement according to claim 3, wherein the magnesium glycerophosphate makes up more than 90% by weight of total magnesium source present in the supplement.

13. The nutritional supplement according to claim 1, containing at least 65% by weight of a mixture of α-lactalbumin and magnesium source.

14. The nutritional supplement according to claim 4, wherein the α-lactalbumin/magnesium source ratio is between 65/35 and 75/25.

15. The nutritional supplemental according to claim 1, comprising 50 to 75% by weight of a mixture of α-lactalbumin and a source of magnesium.

16. The nutritional supplemental according to claim 1, wherein the source of magnesium comprises 4 to 15 mass % magnesium.

17. The nutritional supplemental according to claim 15, wherein the mixture of α-lactalbumin and a source of magnesium comprises 20 wt 50 wt % of the source of magnesium, and said source of magnesium is magnesium glycerophosphate and/or magnesium stearate.

18. The method according to claim 10, comprising administering the nutritional supplement to the animal in a dosage amount of 15 mg to 60 mg per kilo of weight of the animal.

19. The method according to claim 18, comprising administering the supplement orally.

20. The method according to claim 18, wherein the animal is a cat, dog or exotic pet.
